(19) United States
(12) Patent Application Publication
(10) Pub. No.: US 2018/0008598 A1
(43) Pub. Date: Jan. 11, 2018

(54) ANTIBACTERIAL NASAL COMPOSITIONS AND RELATED METHODS

(71) Applicant: Xlear, Inc., Orem, UT (US)

(72) Inventors: Alonzo H. Jones, Plainview, TX (US); Nathan Jones, Orem, UT (US)

(21) Appl. No.: 15/644,596

(22) Filed: Jul. 7, 2017

Related U.S. Application Data
(60) Provisional application No. 62/359,620, filed on Jul. 7, 2016.

(51) Int. Cl. A61K 31/496; A61K 9/00; A61K 31/047; A61K 45/00

(52) U.S. Cl. CPC A61K 31/496; A61K 45/00; A61K 9/0043; A61K 31/047

(57) ABSTRACT

Methods for treatment of infections, such as nasal infections, sinus infections, ear infections, and/or lung infections, along with related compositions. In some implementations, a treatment method may comprise delivering a dose of the composition into a subject's nasal passage to treat the infection. The composition may comprise an antibiotic agent, such as ciprofloxacin. The composition may further comprise at least one non-hexose, sugar alcohol, such as xylitol. Preferably, the dose comprises a sub-clinical dose of the ciprofloxacin or other antibiotic agent.

### RELATED APPLICATIONS

This application claims the benefit under 35 U.S.C. §119(e) of U.S. Provisional Patent Application No. 62/359,620 filed Jul. 7, 2016 and titled “ANTIBACTERIAL NASAL AND SINUS COMPOSITIONS AND RELATED METHODS,” which application is incorporated herein by reference in its entirety.

### SUMMARY

Disclosed herein are methods for treatment of infections, such as nasal infections, sinus infections, ear infections and/or lung infections, along with related antibacterial compositions.

In a specific example of a method for treatment of an infection using a nasal composition, the method may comprise identifying a subject having an infection. The infection may be at least one of a sinus infection, a nasal infection, and a lung infection. A dose of the composition may be delivered into the subject, such as the subject's nasal passage, using, for example, a nasal spray bottle. The composition may comprise an antibiotic and/or antimicrobial agent, such as at least one of ciprofloxacin and ciprofloxacin hydrochloride, and at least one of xylitol and xylose.

In some embodiments and implementations, the dose may comprise a sub-clinical dose of the at least one of ciprofloxacin and ciprofloxacin hydrochloride or other antibiotic. In some such embodiments, the dose may comprise no more than about 150 mg of the at least one of ciprofloxacin and ciprofloxacin hydrochloride. In some such implementations, the dose comprises no more than about 75 mg of the at least one of ciprofloxacin and ciprofloxacin hydrochloride. In some such implementations, the dose comprises between about 20 mg and about 75 mg of the at least one of ciprofloxacin and ciprofloxacin hydrochloride.

In some embodiments and implementations, the composition may comprise between about 0.05 and about 1.0 weight percent of the at least one of ciprofloxacin and ciprofloxacin hydrochloride. In some such implementations, the composition may comprise between about 0.1 and about 1.0 weight percent of the at least one of ciprofloxacin and ciprofloxacin hydrochloride. In some such implementations, the composition may comprise between about 0.1 and about 0.5 weight percent of the at least one of ciprofloxacin and ciprofloxacin hydrochloride.

In some embodiments and implementations, the at least one of xylitol and xylose is present in a concentration effective for providing a synergistic effect with the at least one of ciprofloxacin and ciprofloxacin hydrochloride. In some such implementations, the composition may comprise at least one of xylitol and xylose in a concentration of between about 5 and about 20 weight percent.

In another example of a method for treatment of an infection using a nasal composition, the method may comprise identifying a subject having an infection, and delivering a dose of the composition into the subject's nasal passage. The composition may comprise an antibiotic agent and at least one non-hexose, sugar alcohol, such as at least one of xylitol, xylose, erythritol, erythrose, ribose, ketose, and/or arabinose, which may be present in a concentration of between about 5 and about 20 weight percent. The dose or doses may comprise a sub-clinical dose of the antibiotic agent.

In some implementations, the infection may be at least one of a sinus infection, a nasal infection, and a lung infection.

In some embodiments and/or implementations, the antibiotic agent may comprise at least one of penicillin, amoxicillin, mupirocin, a cephalosporin, ciprofloxacin, ciprofloxacin hydrochloride, and/or vancomycin.

In some embodiments and/or implementations in which the antibiotic agent comprises at least one of ciprofloxacin and ciprofloxacin hydrochloride, the dose may comprise no more than about 75 mg of the at least one of ciprofloxacin and ciprofloxacin hydrochloride.

Some implementations may further comprise delivering a second dose of the composition into the subject's nasal passage.

In some implementations, the step of delivering a dose of the composition into the subject's nasal passage may comprise delivering the dose into the subject's nasal passage using a nasal spray bottle.

In still another example of a method for treatment of an infection using a nasal composition, the method may comprise identifying a subject having an infection, such as at least one of a sinus infection, a nasal infection, an ear infection, and a lung infection, and delivering a dose of the composition into one or both of the subject's nasal passages. The composition may comprise ciprofloxacin in a concentration of between about 0.1 and about 0.5 weight percent and at least one of xylitol and xylose in a concentration of between about 5 and about 20 weight percent. The dose may comprise ciprofloxacin in an amount between about 20 mg and about 75 mg.

The features, structures, steps, or characteristics disclosed herein in connection with one embodiment may be combined in any suitable manner in one or more alternative embodiments.

### DETAILED DESCRIPTION

Antibiotics are often used to treat bacterial infections. However, it has been discovered that antibiotics often have little to no effect on bacterial biofilms. Because chronic nasal infections, sinus infections, ear infections, lung infections and sinusitis are often caused by such biofilms, antibiotic treatment alone is of limited usefulness.

Certain non-hexose sugar alcohols, such as xylitol, xylose, erythritol, ribose, and arabinose, for example, have a sweetness equivalent to that of sucrose, but also possess unique properties that render it unsuitable as a source of energy for certain bacteria and/or other microorganisms. More particularly, without being limited by theory, it is thought that, due to its five-carbon sugar alcohol structure, xylitol, for example, cannot be used as an energy source for most oral microorganisms but may be ingested or otherwise prevent ingestion of other similar energy sources. Regular consumption of xylitol has also been shown to reduce the incidence of dental caries. This is primarily attributed to xylitol's ability to inhibit and/or reduce the growth and acid production of S. mutans, which is thought to be one of the more important bacterium taking part in the pathogenesis of dental caries.

Xylitol has also been demonstrated to inhibit the growth of Streptococcus pneumonia in vitro during its logarithmic growth phase. The Streptococcus pneumonia bacteria species is believed to be the causative agent of certain types of pneumonia and upper respiratory infections, and is also associated with other infectious diseases, such as meningitis and sepsis.

The present inventors have discovered that these unique characteristics make xylitol, and possibly other related sugar alcohols, extremely useful in disrupting bacterial biofilms. Again, without being limited by theory, it is thought that this may be due to the ability of these substances to be treated as a source of energy and taken in by bacteria (due to xylitol's similar shape to other sugars that may serve as an energy source), which leaves no room for six-carbon sugars and thereby impedes bacterial growth and reproduction by, in essence, starvation in the presence of xylitol.

The present inventors have further discovered that, not only may xylitol, and possibly other related non-hexose, flexible sugar alcohols, be useful for starvation of bacteria, but they may also serve as a biofilm disrupter. Without being limited by theory, it is thought that this may happen by starvation of bacteria on the upper layer, after which the layer immediately below is starved, and so on until the biofilm has been suitably disrupted and/or eliminated. The present inventors have further discovered that certain antibiotics may have a synergistic impact on the disruption of biofilms, particularly biofilms in the nasal passages and sinus cavities around the nasal passages. In fact, by providing xylitol or, in some embodiments, other similar sugar alcohols, a much smaller dose (in some cases, a sub-clinical dose, which, in the context of this application, should be interpreted to mean a dose that is less than the concentration/dosage typically required for oral intake) of antibiotics may be needed in order to have a beneficial impact to the patient on such biofilm disruption.

This may have a number of positive benefits. For example, in embodiments comprising a sub-clinical dose of antibiotics, the composition may be sold over the counter. In addition, the combination of xylitol and/or non-hexose sugar alcohols with antibiotics may reduce the ability of the bacteria to evolve resistance to the antibiotic agents. Also, because of the smaller dosage, the potentially harmful impact of the antibiotics on the useful bacteria in the human biodome may be reduced or eliminated.

In addition, because typically free-floating bacteria (planktonic) do not develop resistance, the anti-adherence properties of xylitol and possibly other flexible, non-hexose sugar alcohols, can also provide improved ability to avoid antibiotic resistance. Antibiotic resistance is typically developed in response to quorum sensing. When bacteria are threatened, they release chemical signals and, when the signal gets loud enough, some of the bacteria in the biofilm increase their mutation rate, seeking a way to cope with the threat. Thus, the anti-adherence properties of xylitol, and possibly other related non-hexose, flexible sugar alcohols, may work against the development of antibiotic resistance by disruption of biofilms.

In addition, because of the beneficial properties of xylitol and possibly other flexible, non-hexose sugar alcohols, smaller doses of antibiotics may be used when combined with xylitol than when used apart from xylitol. This may further inhibit development of antibiotic resistance.

It is therefore thought that use of xylitol or, in some embodiments, another non-hexose sugar alcohol, such as xylitol, xylose, erythritol, erythrose, ribose, ketose, and/or arabinose, in conjunction with an antibiotic agent, such as, for example, penicillin, amoxicillin, mupirocin, a cephalosporin (such as cefprozil, cefuroxime axetil, or cephalexin, for example), ciprofloxacin (which may be administered as ciprofloxacin hydrochloride), and/or vancomycin, may result in an improved composition for use in treating infections in the nasal and/or sinus passages, including ear infections (most particularly, inner ear infections). Such compositions may provide a synergistic treatment effect for such conditions, and may also reduce the negative impacts of treating such conditions with a clinical dose of antibiotics alone.

In preferred embodiments and implementations, the concentration and/or dosage of the antibiotic agent may be smaller than would typically be present in a dosage of the same antibiotic agent without the xylitol or other non-hexose sugar alcohols. In some such embodiments and implementations, the concentration and/or dosage may even be sub-clinical. Similarly, some embodiments and implementations may allow for non-systemic application and/or absorption of the antibiotic agent.

The methods and compositions disclosed herein may be particularly useful for treating cystic fibrosis patients. Because lung infections can be quite serious for such patients, providing an improved antimicrobial formulation that both includes ingredients that improve the efficacy of the antimicrobial agent, such as xylitol, and that decrease the possibility of antibiotic resistance, provide a significant improvement in possible treatment of cystic fibrosis and/or its related infections. Similarly, treatment of sinus/nasal infections using the formulations/methods disclosed herein by cystic fibrosis patients may prevent or at least inhibit the development of more serious lung infections.

Moreover, with regard to treatment of cystic fibrosis patients, the xylitol may serve another useful purpose by lowering the sodium concentration in the airway surface fluids of the patient. This liquid typically contains antimicrobial substances that are useful in killing bacteria that are constantly deposited in the lungs. Because the salt concentration in this fluid may inhibit the antimicrobial function of this fluid, and because xylitol can lower this salt concentration, use of xylitol in nasal sprays may contribute to enhancement of the antimicrobial function of the airway surface fluid. When used in combination with relatively low doses of antibiotics, this function may be further enhanced, as explained throughout this disclosure.

In some embodiments and implementations, nasal, ear, lung, and/or sinus infection treatment compositions disclosed herein may comprise methods, agents, compositions, etc. disclosed in U.S. Pat. Nos. 6,054,143 and 6,258,372, both titled “XYLITOL NOSE SPRAY” and U.S. Pat. No. 6,599,883 titled “NASAL DELIVERY OF XYLITOL,” each of which is incorporated herein by reference in its entirety.

Some of the sugar alcohols disclosed herein, such as xylitol in particular, have also been demonstrated to be very effective in moisturizing mucous-lined passages and cavities, such as the nasal and/or sinus passages. Without being limited by theory, this is thought to occur because xylitol can create a hyper-osmotic solution that pulls moisture towards it from surrounding tissues without generated mucous. Thus, some embodiments and implementations disclosed herein may also result in improved ability to add moisture and/or prevent unwanted dryness in nasal and/or sinus passages, along with the accompanying bacterial starvation benefits discussed above.

In some preferred embodiments and implementations, the antibiotic agent may be present in a sub-clinical dosage. For example, with regard to amoxicillin, patients are typically prescribed a dosage of about 500 to about 1,000 mg every 8 hours. Thus, in preferred embodiments and implementations including amoxicillin as the antibiotic agent, amoxicillin would be present in the nasal composition in an amount such that the dosage applied at a particular time contains substantially less than a typical clinical dosage. For example, in some such embodiments and implementations including amoxicillin as the antibiotic agent, amoxicillin would be present in the nasal composition in an amount such that the dosage applied at a particular time (for example, in a single spray or two sprays from a nasal spray containing the composition in each nostril) is no more than about 400 mg. In some such embodiments and implementations including amoxicillin as the antibiotic agent, amoxicillin would be present in the nasal composition in an amount such that the dosage applied at a particular time is between about 50 mg and about 400 mg. In some such embodiments and implementations including amoxicillin as the antibiotic agent, amoxicillin would be present in the nasal composition in an amount such that the dosage applied at a particular time is no more than about 200 mg. In some such embodiments and implementations including amoxicillin as the antibiotic agent, amoxicillin would be present in the nasal composition in an amount such that the dosage applied at a particular time is between about 5 mg and about 100 mg.

In preferred embodiments and implementations including penicillin as the antibiotic agent, penicillin would also preferably be present in the nasal composition in an amount such that the dosage applied at a particular time contains substantially less than a typical clinical dosage. Because penicillin is typically prescribed in lower dosages than amoxicillin, the dosage for embodiments containing penicillin may be slightly lower. For example, in some such embodiments and implementations including penicillin as the antibiotic agent, penicillin would be present in the nasal composition in an amount such that the dosage applied at a particular time (for example, in a single spray or two sprays from a nasal spray containing the composition in each nostril) is no more than about 200 mg. In some such embodiments and implementations including amoxicillin as the antibiotic agent, amoxicillin would be present in the nasal composition in an amount such that the dosage applied at a particular time is between about 50 mg and about 200 mg.

Because ciprofloxacin is typically administered in doses of about 250-750 mg, the dosage for embodiments containing ciprofloxacin may be altered accordingly. For example, in some such embodiments and implementations including ciprofloxacin as the antibiotic agent, ciprofloxacin would be present in the nasal composition in an amount such that the dosage applied at a particular time (for example, in a single spray or two sprays from a nasal spray containing the composition in each nostril) is no more than about 150 mg. In some such embodiments and implementations including ciprofloxacin as the antibiotic agent, ciprofloxacin would be present in the nasal composition in an amount such that the dosage applied at a particular time is no more than about 75 mg. In some such embodiments and implementations including ciprofloxacin as the antibiotic agent, ciprofloxacin would be present in the nasal composition in an amount such that the dosage applied at a particular time is between about 20 mg and about 75 mg. In some embodiments and implementations, including ciprofloxacin as the antibiotic agent, ciprofloxacin would be present in the nasal composition in an amount such that the dosage applied at a particular time is between about 5 mg and about 50 mg.

In terms of the amount of ciprofloxacin in the composition itself, in preferred embodiments and implementations, the composition may include between about 0.05 percent and about 10 percent (weight percent) of ciprofloxacin. In more preferred embodiments and implementations, the composition may include between about 0.05 percent and about 1.0 percent (weight percent) of ciprofloxacin. In more preferred embodiments and implementations, the composition may include between about 0.1% and about 1.0 percent (weight percent) of ciprofloxacin. In more preferred embodiments and implementations, the composition may include between about 0.1% and about 0.5 percent (weight percent) of ciprofloxacin. One or more of these concentrations may be applied in doses that are sub-clinical and/or non-systemic, which, as discussed above, may be achievable because of the synergistic effects of xylitol and/or other related non-hexose, sugar alcohols with the ciprofloxacin.

Similarly, because vancomycin is typically administered in doses even smaller than penicillin, the dosage for embodiments containing vancomycin may be even lower. For example, in some such embodiments and implementations including vancomycin as the antibiotic agent, vancomycin would be present in the nasal composition in an amount such that the dosage applied at a particular time (for example, in a single spray or two sprays from a nasal spray containing the composition in each nostril) is no more than about 100 mg. In some such embodiments and implementations including vancomycin as the antibiotic agent, vancomycin would be present in the nasal composition in an amount such that the dosage applied at a particular time is no more than about 50 mg. In some such embodiments and implementations including vancomycin as the antibiotic agent, vancomycin would be present in the nasal composition in an amount such that the dosage applied at a particular time is between about 10 mg and about 50 mg.

In some preferred embodiments and implementations, a threshold concentration of one or more non-hexose sugar alcohols, such as xylitol, xylose, erythritol, ribose, and/or arabinose, may be used. For example, in some embodiments and implementations, the composition may comprise at least about 0.5% by weight of xylitol, xylose, erythritol, erythrose, ribose, ketose, and/or arabinose. In some such embodiments and implementations, the composition may comprise at least about 2% by weight of xylitol, xylose, erythritol, erythrose, ribose, ketose, and/or arabinose. In some such embodiments and implementations, the composition may comprise between about 0.5% and about 50% by weight of xylitol, xylose, erythritol, erythrose, ribose, ketose, and/or arabinose. In some such embodiments and implementations, the composition may comprise between about 1% and about 30% by weight of xylitol, xylose, erythritol, erythrose, ribose, ketose, and/or arabinose. In some such embodiments and implementations, the composition may comprise between about 5% and about 20% by weight of xylitol, xylose, erythritol, erythrose, ribose, ketose, and/or arabinose.

In certain preferred embodiments and implementations, any of the above-referenced combinations/concentrations of xylitol, xylose, erythritol, erythrose, ribose, ketose, and/or arabinose may be limited to xylitol, xylose, and/or erythritol, which may have some of the most beneficial properties described herein. Similarly, in certain other embodiments and implementations, any of the above-referenced combinations/concentrations of xylitol, xylose, erythritol, erythrose, ribose, ketose, and/or arabinose may be limited to xylitol and/or erythritol. In some such embodiments and implementations, any of the above-referenced combinations/concentrations of xylitol, xylose, erythritol, erythrose, ribose, ketose, and/or arabinose may be limited to xylitol alone.

In certain embodiments and implementations, a solution comprising xylitol, xylose, erythritol, erythrose, ribose, ketose, and/or arabinose and one or more antibiotic agents may further comprise various other ingredients, such as a buffer, a thickening agent, a bioadhesive, and/or a humectant may also be included along with one or more excipients suitable for a pharmaceutical composition.

In embodiments including a buffer, the buffer may be configured to maintain a pH level of the solution. Exemplary suitable buffers include acetate, citrate, and phosphate buffers. The thickening agent may include, for example, one or more of methylcellulose, xanthan gum, carboxyl methylcellulose, polyvinyl alcohol, hydroxpropyl cellulose, carbomer, starches, chitosans, acrylates, and mixtures thereof. In certain embodiments, these substances may also act as suitable bio-adhesives. Suitable exemplary humectants include sorbitol, propylene glycol, glycerol, and/or any combination thereof.

Some embodiments and implementations may further comprise one or more non-pharmaceutical antibacterial agents, such as anise oil or other anise seed extracts, star anise (illicium verum) extracts, propolis, allicin, echinacea, oregano oil, thyme oil, and/or Pau d'arco.

In preferred implementations of methods for treating nasal, ear, and/or sinus infections, the composition may be administered into the nasal passage of a subject, such as by way of a nasal spray or other suitable applicator, at a frequency rate of one or two sprays in each nostril from once daily to four times daily. In a most preferred implementation, the treatment regimen may comprise administration of the composition into the nasal passage of a subject at a frequency rate of one or two sprays in each nostril three times daily, in some such implementations until the bottle is empty.

The preferred dosage range used in connection with one or more the embodiments and/or implementations disclosed herein may be from about 0.1 mL to about 0.6 mL of fluid per dose.

The nasal solution may comprise a nasal spray, and may further comprise a nasal spray bottle configured to deliver the nasal solution. Alternatively, the nasal solution may comprise a nasal dropper configured to deliver the nasal solution in a liquid drop form. In other embodiments, the nasal solution may comprise a gel. In some embodiments, the antibiotic nasal composition may be stored in a nasal spray bottle in a liquid or powder form, and may be distributed into the intranasal pathway and/or nasopharnyx as an aerosol.

It will be understood by those having skill in the art that changes may be made to the details of the above-described embodiments without departing from the underlying principles presented herein. For example, the compositions disclosed herein may be administered via liquid drops from a dropper, topically (in some cases using a cotton swab or the like), orally, via a mister or atomizer, and/or via any other suitable manner of administration. In addition, any suitable combination of various embodiments, or the features thereof, is contemplated.

Any methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment.

It will be apparent to those having skill in the art that changes may be made to the details of the above-described embodiments without departing from the underlying principles set forth herein. The scope of the present inventions should, therefore, be determined only by the following claims.

1. A method for treatment of an infection using a nasal composition, wherein the method comprises the steps of:
identifying a subject having an infection, wherein the infection is at least one of a sinus infection, a nasal infection, and an ear infection;
delivering a dose of the composition into the subject's nasal passage, wherein the composition comprises:
at least one of ciprofloxacin and ciprofloxacin hydrochloride; and
at least one of xylitol and xylose.

2. The method of claim 1, wherein the dose comprises a sub-clinical dose of the at least one of ciprofloxacin and ciprofloxacin hydrochloride.

3. The method of claim 2, wherein the dose comprises no more than about 150 mg of the at least one of ciprofloxacin and ciprofloxacin hydrochloride.

4. The method of claim 3, wherein the dose comprises no more than about 75 mg of the at least one of ciprofloxacin and ciprofloxacin hydrochloride.

5. The method of claim 4, wherein the dose comprises between about 5 mg and about 50 mg of the at least one of ciprofloxacin and ciprofloxacin hydrochloride.

6. The method of claim 4, wherein the composition comprises between about 0.05 and about 1.0 weight percent of the at least one of ciprofloxacin and ciprofloxacin hydrochloride.

7. The method of claim 6, wherein the composition comprises between about 0.1 and about 1.0 weight percent of the at least one of ciprofloxacin and ciprofloxacin hydrochloride.

8. The method of claim 7, wherein the composition comprises between about 0.1 and about 0.5 weight percent of the at least one of ciprofloxacin and ciprofloxacin hydrochloride.

9. The method of claim 1, wherein the at least one of xylitol and xylose is present in a concentration effective for providing a synergistic effect with the at least one of ciprofloxacin and ciprofloxacin hydrochloride.

10. The method of claim 9, wherein the composition comprises at least one of xylitol and xylose in a concentration of between about 5 and about 20 weight percent.

11. A method for treatment of an infection using a nasal composition, wherein the method comprises the steps of:
identifying a subject having an infection;
delivering a dose of the composition into the subject's nasal passage, wherein the composition comprises:
an antibiotic agent; and
at least one non-hexose, sugar alcohol in a concentration of between about 5 and about 20 weight percent, and wherein the dose comprises a sub-clinical dose of the antibiotic agent.

12. The method of claim 11, wherein the infection is at least one of a sinus infection, a nasal infection, and an ear infection.

13. The method of claim 11, wherein the antibiotic agent comprises at least one of penicillin, amoxicillin, mupirocin, a cephalosporin, ciprofloxacin, ciprofloxacin hydrochloride, and/or vancomycin.

14. The method of claim 13, wherein the antibiotic agent comprises at least one of ciprofloxacin and ciprofloxacin hydrochloride.

15. The method of claim 14, wherein the dose comprises no more than about 75 mg of the at least one of ciprofloxacin and ciprofloxacin hydrochloride.

16. The method of claim 11, wherein the at least one non-hexose, sugar alcohol comprises at least one of xylitol, xylose, erythritol, erythrose, ribose, ketose, and/or arabinose.

17. The method of claim 16, wherein the at least one non-hexose, sugar alcohol comprises xylitol.

18. The method of claim 11, further comprising delivering a second dose of the composition into the subject's nasal passage.

19. The method of claim 11, wherein the step of delivering a dose of the composition into the subject's nasal passage comprises delivering the dose into the subject's nasal passage using a nasal spray bottle.

20. A method for treatment of an inner ear infection using a nasal composition, wherein the method comprises the steps of:
identifying a subject having an inner ear infection;
delivering a dose of the composition into the subject's nasal passage, wherein the composition comprises:
ciprofloxacin in a concentration of between about 0.1 and about 0.5 weight percent; and
at least one of xylitol and xylose in a concentration of between about 5 and about 20 weight percent, and wherein the dose comprises:
ciprofloxacin in an amount between about 20 mg and about 75 mg.
